(19) United States
(12) Patent Application Publication
(10) Pub. No.: US 2024/0008483 A1
(43) Pub. Date: Jan. 11, 2024

(54) COMPOSITION FOR MOLD REMEDIATION

(71) Applicant: MOLD GUARD INC., Montreal (CA)

(72) Inventor: Vladimir PODLIPSKIY, San Diego, CA (US)

(21) Appl. No.: 18/252,629

(22) PCT Filed: Nov. 12, 2021
(86) PCT No.: PCT/US2021/059089

§ 371 Date: May 11, 2023

(51) Int. Cl. A01N 37/44; A01N 31/08; A01P 3/00

(52) U.S. Cl. CPC A01N 37/44; A01N 31/08; A01P 3/00

(57) ABSTRACT

The present technology relates to an antifungal composition comprising at least one naturally -occurring compound of the phenylpropanoid family, at least one synthetic derivative of a compound of the phenylpropanoid family, at least one D-isomer of a naturally-occurring aromatic amino acid, at least one synthetic clerk alive of a D-isomer of a naturally-occurring aromatic amino acid, at least one natural monoterpenoid phenol, and at least one halogenated derivative of a natural monoterpenoid phenol. The compositions of the present technology are suitable for use in killing and inhibiting the growth of live cells, spores and mycotoxins associated with fungi. Also provided are methods for killing and inhibiting the growth of the cells, spores and mycotoxins associated with fungi on a surface.

### CROSS-REFERENCE TO RELATED APPLICATION

This application claims the benefit of and priority to U.S. provisional patent application No. 63/113,442, filed on Nov. 13, 2020; the content of which is herein incorporated in entirety by reference.

### FIELD OF TECHNOLOGY

The present technology generally relates to natural antifungal compositions and methods for their use.

### BACKGROUND INFORMATION

Consumer demands for natural plant-based products has been growing in recent years. Natural products are nowadays perceived as equally effective, yet non-toxic, and milder to consumers and the environment and thus superior to their synthetic and chemical counterparts. More specifically, commercial antifungal and/or antimold compositions generally use chemical compounds which are classified as carcinogens or are toxic to humans, animals and other non-target species. Furthermore, these compositions generally work by chemical aggression, meaning that they burn off the mold, and offer no further protection when the product dries.

Some age-old natural antifungal products are presently known and used in the field, however without synthetic modification, these natural compounds are not active enough to effectively, thoroughly and cost efficiently, prevent and kill a wide spectrum of mold.

Therefore, there is a need for natural antifungal compositions which at least overcome some of the above-described problems.

### SUMMARY

Certain aspects and embodiments of the present technology overcome or reduce at least some of the problems of known antifungal compositions.

As aspect of the present technology relates to natural antifungal compositions which form a barrier and protect a surface for at least a year without using corrosive oxidation.

Another aspect of the present technology relates to natural antifungal compositions which comprise synthetic derivatives of readily available natural compounds, which have more potent antifungal properties than their natural counterparts and remain non-toxic.

Another aspect of the present technology relates to a natural antifungal composition that is highly specific and toxic against mold but is safe for humans, is highly efficient against a wide spectrum of fungi and provides long term prophylactic effects against the growth of molds.

Another aspect of the present technology relates to natural antifungal compositions which eradicate or reduce existing fungal growth and prevent and/or suppress the infestation or recurrence of fungus on a surface.

Another aspect of the present technology relates to natural antifungal compositions with enhanced antifungal activity which prevent the growth of a wide spectrum of fungi on a surface.

Another aspect of the present technology relates to natural antifungal compositions that are environmentally and occupationally safe.

A further aspect of the present technology relates to natural antifungal compositions that are capable of protecting humans from excessive exposure to fungi, spores and mycotoxins as a result of infestation in a building.

A yet further aspect of the present technology relates to natural antifungal compositions effective at killing or inhibiting the growth of live cells, spores and mycotoxins associated with a wide spectrum of molds, including black mold, Aspergillus fumigatus, Aspergillus flavus, Aspergillus niger, Stachybotrys chartarum, cladosporium species, fusarium species, and penicillium species.

A yet further aspect of the present technology relates to a natural antifungal composition that is easy to use, simple to manufacture and is comparatively cost effective.

Other aspects and features of the present disclosure will become apparent to those ordinarily skilled in the art upon review of the following description of specific embodiments.

### DETAILED DESCRIPTION

The present disclosure is not limited in its application to the details of construction and the arrangement of components set forth in the following description or illustrated in the drawings. The disclosure is capable of other embodiments and of being practiced or of being carried out in various ways. Also, the phraseology and terminology used herein is for the purpose of description and should not be regarded as limiting.

### Definitions

The use of “including”, “comprising”, or “having”, “containing”, “involving” and variations thereof herein, is meant to encompass the items listed thereafter as well as, optionally, additional items. In the following description, the same numerical references refer to similar elements.

It must be noted that, as used in this specification and the appended claims, the singular form “a”, “an” and “the” include plural referents unless the context clearly dictates otherwise.

As used herein, the term “about” in the context of a given value or range refers to a value or range that is within 20%, preferably within 10%, and more preferably within 5% of the given value or range.

As used herein, the term “and/or” is to be taken as specific disclosure of each of the two specified features or components with or without the other. For example, “A and/or B” is to be taken as specific disclosure of each of (i) A, (ii) B and (iii) A and B, just as if each is set out individually herein.

The recitation herein of numerical ranges by endpoints is intended to include all numbers subsumed within that range (e.g., a recitation of 1 to 5 includes 1, 1.25, 1.33, 1.5, 2, 2.75, 3, 3.80, 4, 4.32, and 5).

As used herein, the term “antifungal” refers to a substance that eradicates or reduces existing fungus growth, and/or prevents, suppresses or inhibits infestation or recurrence of fungus growth.

As used herein, the term “mold” refers microscopic fungi that grow in the form of multicellular filaments, called hyphae.

As used herein, the term “natural” or “naturally occurring” refers to what is found in nature or can be isolated from nature.

As used herein, the term “synthetic” refers to chemically modified.

As used herein, the term “treatment” refers to controlling the growth and spread of microorganisms on a surface, including fungi and fungal matter.

As used herein, the term “prophylaxis” or “prophylactic” refers to guarding from or preventing the spread and/or occurrence of fungus growth.

As used herein, the expression “fungal matter” includes live cells, spores and mycotoxins.

As used herein, the term “emulsion” refers to a mixture of two or more liquids that are normally immiscible.

In one embodiment, the present technology relates to antifungal compositions comprising naturally occurring compounds, having enhanced antifungal activity and long-term preventative effects against the growth and spread of fungal matters. In some instances, the antifungal composition of the present technology is suitable for use in controlling the growth and spread of fungus on a surface.

In one embodiment, the antifungal composition of the present technology comprises at least one naturally-occurring compound of the phenylpropanoid family, at least one synthetic derivative of a compound of the phenylpropanoid family, at least one D-isomer of a naturally-occurring aromatic amino acid, at least one synthetic derivative of a D-isomer of a naturally-occurring aromatic amino acid, at least one natural monoterpenoid phenol, and at least one halogenated derivative of a natural monoterpenoid phenol.

### Phenylpropanoids and Synthetic Derivatives

The phenylpropanoids are a family of plant derived organic compounds with an aromatic ring and a three-carbon propene tail, that are biosynthesized from the amino acid phenylalanine Phenylpropanoids are found throughout the plant kingdom, where they serve as essential components of a number of structural polymers, provide protection from ultraviolet light, defend against herbivores and pathogens, and mediate plant-pollinator interactions as floral pigments and scent compounds. For instance, cinnamic acid is created from phenylalanine by the action of the enzyme phenylalanine ammonia lysase (PAL). A series of enzymatic hydroxylations and methylations leads to coumaric acid, caffeic acid, ferulic acid, 5-hydroxyferulic acid, and sinapic acid. Conversion of these acids to their corresponding esters produces some of the volatile components of herb and flower fragrances. Also, cinnamic aldehydes may be formed by the reduction of the carboxylic acid functional groups in the cinnamic acids. Further reduction provides monolignols including coumaryl alcohol, coniferyl alcohol, and sinapyl alcohol. The monolignols are monomers that may be polymerized to generate various forms of lignin and suberin, which are used as a structural component of plant cell walls. The phenylpropenes, including eugenol, chavicol, safrole and estragole, may be derived from the monolignols. These compounds are the primary constituents of various essential oils. Hydroxylation of cinnamic acid in the 2-position leads to p-coumaric acid, which can be further modified into hydroxylated derivatives such as umbelliferone. Another use of p-coumaric acid via its thioester with coenzyme A (i.e. 4-coumaroyl-CoA), is the production of chalcone. This is achieved with the addition of 3 malonyl-CoA molecules and their cyclization into a second phenyl group (see polyphenols). Chalcone is the precursor of all flavonoids, a diverse class of phytochemicals. Stilbenoids, such as resveratrol, are hydroxylated derivatives of stilbene. They are formed through an alternative cyclization of cinammoyl-CoA or 4-coumaroyl-CoA. Other non-limiting examples of naturally-occurring compounds of the phenylpropanoid family suitable for use in the composition of the present technology, include the apiole, asarone, dillapiole, elemicin, estragole, methyl, eugenol, myristicin. In certain implementations, the phenylpropanoids suitable for the composition of the present technology are selected from D-Phenylalanine, D-Tyrosine, Cinnamic acid and Eugenol, and combinations thereof.

In other embodiments, the antifungal composition of the present technology comprises: i) at least one naturally-occurring compound of the phenylpropanoid family that is selected from D-Phenylalanine, D-Tyrosine, Cinnamic acid and Eugenol, ii) at least one synthetic derivative of a compound of the phenylpropanoid family, iii) at least one D-isomer of a naturally-occurring aromatic amino acid, iv) at least one synthetic derivative of a D-isomer of a naturally-occurring aromatic amino acid, v) at least one natural monoterpenoid phenol, and vi) at least one halogenated derivative of a natural monoterpenoid phenol.

In some embodiments, the least one naturally-occurring compound of the phenylpropanoid family has a molecular weight (MW) ranging between about 100 g/mol and about 400 g/mol, between about 100 g/mol and about 350 g/mol, between about 100 g/mol and about 300 g/mol, between about 100 g/mol and about 250 g/mol, between about 100 g/mol and about 200 g/mol, between about 100 g/mol and about 150 g/mol, between about 150 g/mol and about 400 g/mol, between about 150 g/mol and about 350 g/mol, between about 150 g/mol and about 300 g/mol, between about 150 g/mol and about 250 g/mol, between about 150 g/mol and about 200 g/mol, between about 200 g/mol and about 400 g/mol, between about 200 g/mol and about 350 g/mol, between about 200 g/mol and about 300 g/mol, between about 200 g/mol and about 250 g/mol, between about 250 g/mol and about 400 g/mol, between about 250 g/mol and about 350 g/mol, between about 250 g/mol and about 300 g/mol, between about 300 g/mol and about 400 g/mol, between about 300 g/mol and about 350 g/mol, or between about 350 g/mol and about 400 g/mol.

In certain embodiments, the composition of the present technology may comprise a combination of naturally-occurring compounds of the phenylpropanoid family, having different molecular weights.

In some embodiments, the antifungal composition of the present technology further comprises at least one synthetic derivative of a compound within the phenylpropanoid family. In some instances, the at least one synthetic derivative of a compound within the phenylpropanoid family is at least one synthetic phenylalanine derivative, at least one synthetic tyrosine derivative or combinations thereof. In other instances, the at least one synthetic phenylalanine derivative is selected from D-Phenylalanine methyl ester, 2-nitro-D-Phenylalanine, pentafluoro-D-Phenylalanine, and 2-chloro-D-Phenylalanine, and combinations thereof. In yet other instances, the at least one synthetic tyrosine derivative is selected from N-acethyl-D-Tyrosine, D-Tyrosine methyl ester, and D-Tyrosine ethyl ester and combinations thereof.

Advantageously, the inventors of the present technology have discovered that synthetic derivatives of phenylalanine and tyrosine such as D-Phenylalanine methyl ester, 2-nitro-D-Phenylalanine, pentafluoro-D-Phenylalanine, 2-chloro-D-Phenylalanine, N-acethyl-D-Tyrosine, D-Tyrosine methyl ester, and D-Tyrosine ethyl ester have antifungal properties and can thus be used as active antifungal agent in the composition of the present technology.

In other embodiments, the antifungal composition of the present technology comprises: i) at least one naturally-occurring compound of the phenylpropanoid family that is selected from D-Phenylalanine, D-Tyrosine, Cinnamic acid and Eugenol, ii) at least one synthetic derivative of a compound of the phenylpropanoid family that is selected from D-Phenylalanine methyl ester, 2-nitro-D-Phenylalanine, pentafluoro-D-Phenylalanine, 2-chloro-D-Phenylalanine, N-acetyl-D-Tyrosine, D-Tyrosine methyl ester, and D-Tyrosine ethyl ester , iii) at least one D-isomer of a naturally-occurring aromatic amino acid, iv) at least one synthetic derivative of a D-isomer of a naturally-occurring aromatic amino acid, v) at least one natural monoterpenoid phenol, and vi) at least one halogenated derivative of a natural monoterpenoid phenol.

In some embodiments, the antifungal composition of the present technology comprises at least one synthetic derivative of a compound within the phenylpropanoid family having a molecular weight (MW) ranging between about 150 g/mol and about 1400 g/mol, between about 150 g/mol and about 1300 g/mol, between about 150 g/mol and about 1200 g/mol, between about 150 g/mol and about 1100 g/mol, between about 150 g/mol and about 1000 g/mol, between about 150 g/mol and about 900 g/mol, between about 150 g/mol and about 800 g/mol, between about 150 g/mol and about 700 g/mol, between about 150 g/mol and about 600 g/mol, between about 150 g/mol and about 500 g/mol, between about 150 g/mol and about 400 g/mol, between about 150 g/mol and about 300 g/mol, between about 300 g/mol and about 1400 g/mol, between about 300 g/mol and about 1300 g/mol, between about 300 g/mol and about 1200 g/mol, between about 300 g/mol and about 1100 g/mol, between about 300 g/mol and about 1000 g/mol, between about 300 g/mol and about 900 g/mol, between about 300 g/mol and about 800 g/mol, between about 300 g/mol and about 700 g/mol, between about 300 g/mol and about 600 g/mol, between about 300 g/mol and about 500 g/mol, between about 300 g/mol and about 400 g/mol, 400 g/mol and about 1400 g/mol, between about 400 g/mol and about 1300 g/mol, between about 400 g/mol and about 1200 g/mol, between about 400 g/mol and about 1100 g/mol, between about 400 g/mol and about 1000 g/mol, between about 400 g/mol and about 900 g/mol, between about 400 g/mol and about 800 g/mol, between about 400 g/mol and about 700 g/mol, between about 400 g/mol and about 600 g/mol, between about 400 g/mol and about 500 g/mol, between about 500 g/mol and about 1400 g/mol, between about 500 g/mol and about 1300 g/mol, between about 500 g/mol and about 1200 g/mol, between about 500 g/mol and about 1100 g/mol, between about 500 g/mol and about 1000 g/mol, between about 500 g/mol and about 900 g/mol, between about 500 g/mol and about 800 g/mol, between about 500 g/mol and about 700 g/mol, between about 500 g/mol and about 600 g/mol, between about 600 g/mol and about 1400 g/mol, between about 600 g/mol and about 1300 g/mol, between about 600 g/mol and about 1200 g/mol, between about 600 g/mol and about 1100 g/mol, between about 600 g/mol and about 1000 g/mol, between about 600 g/mol and about 900 g/mol, between about 600 g/mol and about 800 g/mol, between about 600 g/mol and about 700 g/mol, between about 700 g/mol and about 1400 g/mol, between about 700 g/mol and about 1300 g/mol, between about 700 g/mol and about 1200 g/mol, between about 700 g/mol and about 1100 g/mol, between about 700 g/mol and about 1000 g/mol, between about 700 g/mol and about 900 g/mol, between about 700 g/mol and about 800 g/mol, between about 800 g/mol and about 1400 g/mol, between about 800 g/mol and about 1300 g/mol, between about 800 g/mol and about 1200 g/mol, between about 800 g/mol and about 1100 g/mol, between about 800 g/mol and about 1000 g/mol, between about 800 g/mol and about 900 g/mol, between about 900 g/mol and about 1400 g/mol, between about 900 g/mol and about 1300 g/mol, between about 900 g/mol and about 1200 g/mol, between about 900 g/mol and about 1100 g/mol, between about 900 g/mol and about 1000 g/mol, between about 1000 g/mol and about 1400 g/mol, between about 1000 g/mol and about 1300 g/mol, between about 1000 g/mol and about 1200 g/mol, between about 1000 g/mol and about 1100 g/mol, between about 1100 g/mol and about 1400 g/mol, between about 1100 g/mol and about 1300 g/mol, between about 1100 g/mol and about 1200 g/mol, between about 1200 g/mol and about 1400 g/mol, between about 1200 g/mol and about 1300 g/mol, or between about 1300 g/mol and about 1400 g/mol.

In certain embodiments, the at least one synthetic derivative of a compound within the phenylpropanoid family, is a synthetic derivative of the phenylpropanoid compound used in the composition of the present technology. In some instances, wherein a combination of phenylpropanoid compounds are used, the composition of the present technology may comprise the synthetic derivative of a single phenylpropanoid compound used in the composition or may comprise a combination of synthetic derivates derived from the phenylpropanoid compounds used. In further instances, wherein more than one synthetic derivative of a phenylpropanoid compound is used in the composition, the synthetic derivates of the phenylpropanoid compounds may have different molecular weights.

In certain embodiments, the proportion of the at least one naturally-occurring compound of the phenylpropanoid family, and the at least one synthetic derivative of a compound of the phenylpropanoid family in the composition is between about 0.01% and about 5.0 w/w, between about 0.01% and about 4.0% w/w, between about 0.01% and about 3.0% w/w, between about 0.01% and about 2.0% w/w, between about 0.01% and about 1.0% w/w, between about 0.01% and about 0.5% w/w, between about 0.5% and about 5.0 w/w, between about 0.5% and about 4.0% w/w, between about 0.5% and about 3.0% w/w, between about 0.5% and about 2.0% w/w, between about 0.5% and about 1.0% w/w, between about 1.0% and about 5.0 w/w, between about 1.0% and about 4.0% w/w, between about 1.0% and about 3.0% w/w, between about 1.0% and about 2.0% w/w, between about 2.0% and about w/w, between about 2.0% and about 4.0% w/w, between about 2.0% and about 3.0% w/w, between about 3.0% and about 5.0 w/w, between about 3.0% and about 4.0% w/w, between about 3.0% and about 5.0 w/w, between about 3.0% and about 4.0% w/w, or between about 4.0% and about 5.0 w/w.

### D-Isomers of Naturally-Occurring Aromatic Amino Acids and Synthetic Derivatives

D-isomers of amino acids (also referred to as D-aromatic amino acid) are where the stereogenic carbon alpha to the amino group has the D-configuration. For most naturally-occurring amino acids, this carbon has the L-configuration. L- and D-amino acids have identical properties (color, solubility, melting point) under many conditions. In biological contexts however, which are chiral, these enantiomers can behave very differently. D-isomers of naturally-occurring aromatic amino acid include D-phenylalanine (DPA), D-tryptophan, and D-tyrosine, and D-histidine.

In certain embodiments, the D-isomer of naturally-occurring aromatic amino acid used in the composition of the present technology is D-tyrosine.

In other embodiments the D-isomer of naturally-occurring aromatic amino acid is DPA.

In other embodiments, the antifungal composition of the present technology comprises i) at least one naturally-occurring compound of the phenylpropanoid family that is selected from D-Phenylalanine, D-Tyrosine, Cinnamic acid and Eugenol, ii) at least one synthetic derivative of a compound of the phenylpropanoid family that is selected from D-Phenylalanine methyl ester, 2-nitro-D-Phenylalanine, pentafluoro-D-Phenylalanine, 2-chloro-D-Phenylalanine, N-acethyl-D-Tyrosine, D-Tyrosine methyl ester, and D-Tyrosine ethyl ester, iii) at least one D-isomer of a naturally-occurring aromatic amino acid that is selected from D-tyrosine and D-phenylalanine, iv) at least one synthetic derivative of a D-isomer of a naturally-occurring aromatic amino acid, v) at least one natural monoterpenoid phenol, and vi) at least one halogenated derivative of a natural monoterpenoid phenol.

In some embodiments, the antifungal composition of the present technology comprises at least one D-isomer of a naturally-occurring aromatic amino acid having a molecular weight (MW) ranging between about 150 g/mol and about 250 g/mol, between about 150 g/mol and about 200 g/mol, or between about 200 g/mol and about 250 g/mol.

The antifungal composition of the present technology further comprises at least one synthetic derivative of a D-isomer of a naturally-occurring aromatic amino acid. Advantageously, D-isomers of a naturally-occurring aromatic amino acid and their synthetic derivates have antifungal properties. Without being bound by theory, since D-isomers of a naturally-occurring aromatic amino acid and their synthetic derivates are hydrophobic, it is believed that they remain on treated surfaces for extended periods of time and thus exert long lasting antifungal prophylactic effects. In certain embodiments, the synthetic derivative of the D-isomer of a naturally-occurring aromatic amino acid may be a methyl ester derivative of the D-isomer of a naturally-occurring aromatic amino acid. Advantageous, and without being bound by theory, methyl esters are believed to be more hydrophobic compared to their relative acid or other synthetic forms and may thus result in long lasting antifungal prophylactic effects.

In certain implementation of this embodiment, therefore, the synthetic derivative of the D-isomer of a naturally-occurring aromatic amino acid is D-tyrosine-methyl-ester, or optionally D-phenylalanine-methyl-ester.

In other embodiments, the antifungal composition of the present technology comprises: i) at least one naturally-occurring compound of the phenylpropanoid family that is selected from D-Phenylalanine, D-Tyrosine, Cinnamic acid and Eugenol, ii) at least one synthetic derivative of a compound of the phenylpropanoid family that is selected from D-Phenylalanine methyl ester, 2-nitro-D-Phenylalanine, pentafluoro-D-Phenylalanine, 2-chloro-D-Phenylalanine, N-acetyl-D-Tyrosine, D-Tyrosine methyl ester, and D-Tyrosine ethyl ester, iii) at least one D-isomer of a naturally-occurring aromatic amino acid that is selected from D-tyrosine and D-phenylalanine, iv) at least one synthetic derivative of the D-isomer of a naturally-occurring aromatic amino acid that is selected from D-tyrosine-methyl-ester, and D-phenylalanine-methyl-ester, v) at least one natural monoterpenoid phenol, and vi) at least one halogenated derivative of a natural monoterpenoid phenol.

In some embodiments, the molecular weight of the at least one synthetic derivative of the D-isomer of a naturally-occurring aromatic amino acid is between about 160 g/mol and about 700 g/mol, between about 160 g/mol and about 600 g/mol, between about 160 g/mol and about 500 g/mol, between about 160 g/mol and about 400 g/mol, between about 160 g/mol and about 300 g/mol, between about 300 g/mol and about 700 g/mol, between about 300 g/mol and about 600 g/mol, between about 300 g/mol and about 500 g/mol, between about 300 g/mol and about 400 g/mol, between about 400 g/mol and about 700 g/mol, between about 400 g/mol and about 600 g/mol, between about 400 g/mol and about 500 g/mol, between about 500 g/mol and about 700 g/mol, between about 500 g/mol and about 600 g/mol, or between about 600 g/mol and about 700 g/mol.

In certain embodiments, the at least one synthetic derivative of a D-isomer of a naturally-occurring aromatic amino acid, is a synthetic derivative of the D-aromatic amino acid used in the composition. In other embodiments, wherein a combination of D-aromatic amino acids are used, the composition may comprise the synthetic derivative of a single D-aromatic amino acid used in the composition, or may comprise a combination of synthetic derivatives derived from the various D-aromatic amino acids used. In further embodiments, wherein more than one synthetic derivative of a D-aromatic amino acid is used in the composition, the synthetic derivatives of the D-aromatic amino acids may have different molecular weights.

In other embodiments, the proportion of the at least one D-isomer of a naturally-occurring aromatic amino acid and the at least one synthetic derivative of a D-isomer of a naturally-occurring aromatic amino acid in the composition is between about 0.01% and about 5% w/w, between about and about 4.0% w/w, between about 0.01% and about 3.0% w/w, between about 0.01% and about 2.0% w/w, between about 0.01% and about 1.0% w/w, between about 0.01% and about 0.5% w/w, between about 0.5% and about 5.0% w/w, between about 0.5% and about 4.0% w/w, between about 0.5% and about 3.0% w/w, between about 0.5% and about 2.0% w/w, between about 0.5% and about 1.0% w/w, between about 1.0% and about 5.0% w/w, between about 1.0% and about 4.0% w/w, between about 1.0% and about 3.0% w/w, between about 1.0% and about 2.0% w/w, between about 2.0% and about 5.0% w/w, between about 2.0% and about 4.0% w/w, between about 2.0% and about 3.0% w/w, between about 3.0% and about 5.0% w/w, between about 3.0% and about 4.0% w/w, between about 3.0% and about 5.0% w/w, between about 3.0% and about 4.0% w/w, or between about 4.0% and about 5.0% w/w.

### Natural Monoterpenoid Phenols and Synthetic Derivatives

Natural monoterpenoid phenols are generally isolated and purified from plant oil extracts. Non-limiting examples of monoterpenoid phenols suitable for use in the composition of the present technology may include, thymol, carvacrol, eucalyptol, eugenol and the like. Thymol (isopropyl-cresol) is one particularly suitable monoterpene phenol, which is a crystalline substance that has a boiling point of about 238° C. at atmospheric pressure. Carvacrol (isopropyl-o-cresol), an isomer of thymol, is another suitable compound. Carvacrol is a liquid with a boiling point of about 233° C. at atmospheric pressure.

In certain embodiments of the composition of the present technology, the natural monoterpenoid phenol is thymol.

In other embodiments, the antifungal composition of the present technology comprises: i) at least one naturally-occurring compound of the phenylpropanoid family that is selected from D-Phenylalanine, D-Tyrosine, Cinnamic acid and Eugenol, ii) at least one synthetic derivative of a compound of the phenylpropanoid family that is selected from D-Phenylalanine methyl ester, 2-nitro-D-Phenylalanine, pentafluoro-D-Phenylalanine, 2-chloro-D-Phenylalanine, N-acethyl-D-Tyrosine, D-Tyrosine methyl ester, and D-Tyrosine ethyl ester, iii) at least one D-isomer of a naturally-occurring aromatic amino acid that is selected from D-tyrosine and D-phenylalanine, iv) at least one synthetic derivative of the D-isomer of a naturally-occurring aromatic amino acid that is selected from D-tyrosine-methyl-ester, and D-phenylalanine-methyl-ester, v) thymol, and vi) at least one halogenated derivative of a natural monoterpenoid phenol.

In addition to being employed in an isolated form, essential oils comprising monoterpene phenols as major constituents may also be employed, with the final concentrations of the monoterpene phenols being within the ranges provided herein. The term “major constituent” generally refers to those essential oils having monoterpene phenols in an amount of more than about 50 wt. %. It is well-known in the art that such essential oils may also contain lesser amounts of other constituents, such as non-aromatic terpene compounds. Essential oils comprising monoterpenoid phenols as major constituents may include, but are not limited to, anise oil, bay oil terpineless, clove bud, clove leaf, clove oil, clove stem, origanum oil, peru balsam, pimento oil, eucalyptus oil, thyme oil and mixtures thereof. In some embodiments, a molecular weight (MW) of the at least one natural monoterpenoid phenol used in the antifungal composition of the present technology is between about 2 g/mol and about 100 g/mol, between about 2 g/mol and about 80 g/mol, between about 2 g/mol and about 60 g/mol, between about 2 g/mol and about 40 g/mol, between about 2 g/mol and about 20 g/mol, between about 2 g/mol and about 10 g/mol, between about 10 g/mol and about 100 g/mol, between about 10 g/mol and about 80 g/mol, between about 10 g/mol and about 60 g/mol, between about 10 g/mol and about 40 g/mol, between about 10 g/mol and about 20 g/mol, between about 20 g/mol and about 100 g/mol, between about 20 g/mol and about 80 g/mol, between about 20 g/mol and about 60 g/mol, between about 20 g/mol and about 40 g/mol, between about 40 g/mol and about 100 g/mol, between about 40 g/mol and about 80 g/mol, between about 140 g/mol and about 60 g/mol, between about 60 g/mol and about 100 g/mol, between about 60 g/mol and about 80 g/mol, or between about 80 g/mol and about 100 g/mol.

In certain embodiments, the composition of the present technology may comprise a combination of natural monoterpenoid phenols, having different molecular weights.

In other embodiments, the composition may comprise a mixture of essential oils having a single or a combination of natural monoterpenoid phenols as their major constituent. In these embodiments, the natural monoterpenoid phenols may also have different molecular weights.

The antifungal composition of the present technology further comprises at least one halogenated derivative of a natural monoterpenoid phenol. Halogenated derivatives of phenols generally demonstrate enhanced bioactivities. In certain embodiments, the natural monoterpenoid phenol may be halogenated at a single position on the phenol. In other embodiments, the natural monoterpenoid phenol may be halogenated at more than one position on the phenol. The halogen may be fluorine, chlorine, bromine, iodine and astatine or any combinations thereof depending on the embodiment. In embodiments wherein the monoterpenoid phenol is halogenated at more than one position, the halogen may be the same or different at the various positions on the phenol. In certain embodiments, the halogenated derivative of the natural monoterpenoid phenol may be synthetic. In yet other embodiments, the halogenated derivative of the natural monoterpenoid phenol may be isolated from a natural source, such as from marine organisms or insects. Non limiting examples of halogenated derivatives of natural monoterpenoid phenols suitable for use in the composition of the present technology include chlorothymol, iodothymol, bromothymol, chlorocarvacrol, iodocarvacrol, bromocarvacrol, chloroeugenol, iodoeugenol and bromoeugenol.

In certain embodiments, any combination of halogenated derivatives of natural monoterpenoid phenols may be use in the composition. In other embodiments, the halogenated derivative of the natural monoterpenoid phenol used in the composition of the present technology is 4-chlorothymol, or optionally 4-iodothymol. In other embodiments, the composition of the present technology comprises: i) at least one naturally-occurring compound of the phenylpropanoid family that is selected from D-Phenylalanine, D-Tyrosine, Cinnamic acid and Eugenol, ii) at least one synthetic derivative of a compound of the phenylpropanoid family that is selected from D-Phenylalanine methyl ester, 2-nitro-D-Phenylalanine, pentafluoro-D-Phenylalanine, 2-chloro-D-Phenylalanine, N-acetyl-D-Tyrosine, D-Tyrosine methyl ester, and D-Tyrosine ethyl ester, iii) at least one D-isomer of a naturally-occurring aromatic amino acid that is selected from D-tyrosine and D-phenylalanine, iv) at least one synthetic derivative of the D-isomer of a naturally-occurring aromatic amino acid that is selected from D-tyrosine-methyl-ester, and D-phenylalanine-methyl-ester, v) thymol, and vi) at least one halogenated derivative of a natural monoterpenoid phenol selected from 4-chlorothynol and 4-iodothymol.

In some embodiments, a molecular weight (MW) of at least one halogenated derivative of a natural monoterpenoid phenol used in the antifungal composition of the present technology is between about 5 g/mol and about 130 g/mol, between about 5 g/mol and about 120 g/mol, between about 5 g/mol and about 100 g/mol, between about 5 g/mol and about 80 g/mol, between about 5 g/mol and about 60g/mol, between about 5 g/mol and about 40 g/mol, between about 5 g/mol and about 20 g/mol, between about 5 g/mol and about 10 g/mol, between about 10 g/mol and about 130 g/mol, between about 10 g/mol and about 120 g/mol, between about 10 g/mol and about 100 g/mol, between about 10 g/mol and about 80 g/mol, between about 10 g/mol and about 60 g/mol, between about 10 g/mol and about 40 g/mol, between about 10 g/mol and about 20 g/mol, between about 20 g/mol and about 130 g/mol, between about 20 g/mol and about 120 g/mol, between about 20 g/mol and about 100 g/mol, between about 20 g/mol and about 80 g/mol, between about 20 g/mol and about 60 g/mol, between about 20 g/mol and about 40 g/mol, between about 40 g/mol and about 130 g/mol, between about 40 g/mol and about 120 g/mol, between about 40 g/mol and about 100 g/mol, between about 40 g/mol and about 80 g/mol, between about 40 g/mol and about 60 g/mol, between about 60 g/mol and about 130 g/mol, between about 60 g/mol and about 120 g/mol, between about 60 g/mol and about 100 g/mol, between about 60 g/mol and about 80 g/mol, between about 80 g/mol and about 130 g/mol, between about 80 g/mol and about 120 g/mol, between about 80 g/mol and about 100 g/mol, between about 100 g/mol and about 130 g/mol, between about 100 g/mol and about 120 g/mol, or between about 120 g/mol and about 130 g/mol.

In certain embodiments, the at least one halogenated derivative of the natural monoterpenoid phenol, is a halogenated derivative of the monoterpenoid phenol used in the composition. In other embodiments, wherein a combination of natural monoterpenoid phenols are used, the composition may comprise the halogenated derivative of a single natural monoterpenoid phenol or may comprise a combination of halogenated derivatives derived from the various natural monoterpenoid phenol used in the composition. In further embodiments, wherein more than one halogenated derivative of a natural monoterpenoid phenol is used in the composition, the halogenated derivatives of the natural monoterpenoid phenols may have different molecular weights.

In other embodiments, the proportion of the at least one natural monoterpenoid phenol and the at least one halogenated derivative of a natural monoterpenoid phenol in the composition is between about 0.1% and about 10% w/w, between about 0.1% and about 9.0% w/w, between about 0.1% and about 8.0% w/w, between about 0.1% and about 7.0% w/w, between about 0.1% and about 6.0% w/w, 30 between about 0.1% and about 5.0% w/w, between about 0.1% and about 4.0% w/w, between about 0.1% and about 3.0% w/w, between about 0.1% and about 2.0% w/w, between about 0.1% and about 1.0% w/w, between about 1.0% and about 10% w/w, between about 1.0% and about 9.0% w/w, between about 1.0% and about 8.0% w/w, between about 1.0% and about 7.0% w/w, between about 1.0% and about 6.0% w/w, between about 1.0% and about 5.0% w/w, between about 1.0% and about 4.0% w/w, 35 between about 1.0% and about 3.0% w/w, between about 1.0% and about 2.0% w/w, between about 2.0% and about 10% w/w, between about 2.0% and about 9.0% w/w, between about 2.0% and about 8.0% w/w, between about 2.0% and about 7.0% w/w, between about 2.0% and about 6.0% w/w, between about 2.0% and about 5.0% w/w, between about 2.0% and about 4.0% w/w, between about 2.0% and about 3.0% w/w, between about 3.0% and about 10% w/w, between about 3.0% and about 9.0% w/w, between about 3.0% and about 8.0% w/w, between about 3.0% and about 7.0% w/w, between about 3.0% and about 6.0% w/w, between about 3.0% and about 5.0% w/w, between about 3.0% and about 4.0% w/w, between about 4.0% and about 10% w/w, between about 4.0% and about 9.0% w/w, between about 4.0% and about 8.0% w/w, between about 4.0% and about 7.0% w/w, between about 4.0% and about 6.0% w/w, between about 4.0% and about 5.0% w/w, between about and about 10% w/w, between about 5.0% and about 9.0% w/w, between about 5.0% and about 8.0% w/w, between about 5.0% and about 7.0% w/w, between about 5.0% and about 6.0% w/w, between about 6.0% and about 10% w/w, between about 6.0% and about 9.0% w/w, between about 6.0% and about 8.0% w/w, between about 6.0% and about 7.0% w/w, between about 7.0% and about 10% w/w, between about 7.0% and about 9.0% w/w, between about 7.0% and about 8.0% w/w, between about 8.0% and about 10% w/w, between about 8.0% and about 9.0% w/w, or between about 9.0% and about 10% w/w.

### Additional Components

In certain embodiments, the composition of the present technology comprises additional components. Examples of additional components that may be present in the compositions are surfactants. Surfactants are generally used to lower the surface tension between two liquids, between a gas and a liquid, or between a liquid and a solid and may act as detergents, wetting agents, emulsifiers, foaming agents, or dispersants. Surfactants are usually organic compounds that are amphiphilic, meaning they contain both hydrophobic groups (their tails) and hydrophilic groups (their heads) and are classified into: anionic, cationic, zwitterionic or nonionic depending on the composition of their head groups.

In certain embodiments, the proportion of the at least one surfactant in the composition of the present technology may be between about 0.1% to about 5.0% w/w, between about 0.1% to about 4.0% w/w, between about 0.1% to about 3.0% w/w, between about 0.1% to about 2.0% w/w, between about to about 1.0% w/w, between about 0.1% to about 0.5% w/w, between about 0.5% to about 5.0% w/w, between about 0.5% to about 4.0% w/w, between about 0.5% to about 3.0% w/w, between about 0.5% to about 2.0% w/w, between about 0.5% to about 1.0% w/w, between about 1.0% to about 5.0% w/w, between about 1.0% to about 4.0% w/w, between about 1.0% to about 3.0% w/w, between about 1.0% to about 2.0% w/w, between about 2.0% to about 5.0% w/w, between about 2.0% to about 4.0% w/w, between about 2.0% to about 3.0% w/w, between about 3.0% to about 5.0% w/w, between about 3.0% to about 4.0% w/w, or between about 4.0% to about 5.0% w/w.

In certain embodiments, the surfactant used in the composition of the present technology may be a non-ionic surfactant. These surfactants do not bear an electric charge and thus do not interact with calcium and magnesium ions in hard water. The major group of non-ionic surfactants are the ethoxylates, made by condensing long chain alcohols with epoxyethane to form ethers. The long chain alcohol can come from either a synthetic or natural source. The hydrophilic properties of the ethoxylates is generally conferred by the presence of a number of oxygen atoms in the molecule which form hydrogen bonds with water. Generally, non-ionic surfactants are considered to be more surface active and be better emulsifiers than anionic surfactants at similar concentrations. In certain implementations of these embodiments, the non-ionic surfactant is a polyethoxylated non-ionic surfactant. In further implementation of these embodiments, the polyethoxylated non-ionic surfactant comprises 6 ethoxylated groups. Other suitable non-ionic surfactants include, but are not limited to, alkoxylated alcohols or ethers; alkyl ethoxylates; alkylamido ethoxylates; alkyl glucosides; alkoxylated carboxylic acids; sorbitan derivatives where the alkyl chain length varies from 8 to 24, or the like, for example, nonylphenol ethoxylate-3; alkyl ethoxylates-3; oleyl carboxylic diethylamides; and the like and mixtures thereof.

In other embodiments, the composition of the present technology comprises at least one polymer. Polymers suitable for use in the composition of the present technology may be of synthetic or natural origin. In certain implementations of this embodiment, the polymer can act as a surfactant. Polymeric surfactants are surfactants that consist of polymer in both their head and tail groups. Advantageously, polymeric surfactants form micelles at much lower concentrations than other low-molecular surfactants. Furthermore, polymeric surfactants are effective at reducing the interfacial tension between oil and water in oil/water emulsions and thus promote the formation of smaller droplets and thereby stabilize aqueous and non-aqueous emulsions. Non-limiting examples of synthetic polymers suitable for use in the composition of the present technology include Polyvinylpyrrolidone (PVP), PEG-Alkyd, A-B-A Tri-Block copolymer, oligomeric, comb graft copolymers, and mixtures thereof. In a preferred implementation of this embodiment, PVP is used as a synthetic polymeric surfactant.

In other implementations of these embodiments, natural polymers may be used as surfactants in the composition of the present technology. Non-limiting examples of natural polymers suitable for use in the composition of the present technology include alginates, pectins, protein-based product, chitosans and the like. In a preferred implementation of this embodiment, chitosan is used as a natural polymeric surfactant. In other embodiments, combinations of natural and synthetic polymeric surfactants may be used in the composition. For example, in some implementations a mixture of PVP and chitosan may be used.

In other embodiments, the composition of the present technology may further comprise at least one plant oil extract. In certain implementation of this embodiment, the plant oil extract may act as a surfactant. Non-limiting examples of plant oils suitable for use as surfactants in the composition of the present technology include tea tree oil, eucalyptus oil, canola oil, castor oil, soybean oil and the like. In further embodiments, the composition of the present technology may comprise any combination of the surfactants listed above, acting together as co-surfactants. Optionally, the surfactants may be biodegradable.

In some embodiments, the composition of the present technology may further comprise at least one additive selected from minerals, emulsifiers, defoamers, thickeners, dispersants, stabilizers, suspending agents, adjuvants, preservatives, polymers, acids, bases, dyes, antifreezes, biocides, fillers, wetting agents, or solvents/dilutants. Suitable substances are those which can be typically be used for the treatment of surfaces and are compatible with the composition.

The proportion of the additives in the composition may be between about 5% and about 90% depending on the proportions of each of the at least one naturally-occurring compound within the phenylpropanoid family, the at least one synthetic derivative of a compound within the phenylpropanoid family, the at least one D-isomer of a naturally-occurring aromatic amino acid, the at least one synthetic derivative of a D-isomer of a naturally-occurring aromatic amino acid, the at least one natural monoterpenoid phenol, or the at least one halogenated derivative of a natural monoterpenoid phenol present in a particular embodiment. In some embodiments, the additive may be a diluent. In some implementations of this embodiment, the diluent may be water. In further implementations, the diluent may be an ester or ethyl lactate. The ester may be synthetic or natural. In one embodiment, the diluent is a naturally occurring ester such as ethyl lactate, ethyl citrate, triethyl citrate, or the like.

In certain embodiments, any one or more of the at least one naturally-occurring compound within the phenylpropanoid family, the at least one synthetic derivative of a compound within the phenylpropanoid family, the at least one D-isomer of a naturally-occurring aromatic amino acid, the at least one synthetic derivative of a D-isomer of a naturally-occurring aromatic amino acid, the at least one natural monoterpenoid phenol, or the at least one halogenated derivative of a natural monoterpenoid phenol may have antifungal properties.

In other embodiments, any one or more of the at least one naturally-occurring compound within the phenylpropanoid family, the at least one synthetic derivative of a compound within the phenylpropanoid family, the at least one D-isomer of a naturally-occurring aromatic amino acid, the at least one synthetic derivative of a D-isomer of a naturally-occurring aromatic amino acid, the at least one natural monoterpenoid phenol, or the at least one halogenated derivative of a natural monoterpenoid phenol may have antimold properties including anti-sporulation, and/or anti-mycotoxin properties.

Molds do not form a specific taxonomic or phylogenetic grouping, but can be found in the divisions Zygomycota, Deuteromycota and Ascomycota. There are thousands of known species of molds, which include opportunistic pathogens, exclusive saprotrophs, aquatic species and thermophiles. Like all fungi, molds derive energy not through photosynthesis but from the organic matter on which they live. In order to reproduce, molds create spores that can be carried by air currents. The formation and release of spores from molds is referred to as sporulation. When these spores land on a surface they need only three things to grow into mold: nutrients, moisture, and time. Due to their size, some spores can remain airborne indefinitely and mold spores are a common component of household and workplace dust. When mold spores are present in large quantities, they can present a health hazard to humans. Many molds also secrete mycotoxins to inhibit the growth of competing microorganisms. Importantly, some mycotoxins pose serious health risks to humans and animals.

In certain embodiments, the antifungal and/or antimold properties of the composition of the present technology may be associated with an ability to inhibit any one or more of the growth of live fungal cells, the growth of spores into fungi or to directly kill live fungal cells. The antifungal and/or antimold properties of the composition may also be associated with an ability to inhibit sporulation, and/or inhibit the release of mycotoxins from molds or fungi.

In certain embodiments, the antimold properties of the composition of the present technology is against the Aspergillus genus of fungi. The Aspergillus genus of fungi are molds, of which the Aspergillus fumigatus, Aspergillus flavus, and Aspergillus niger are known to be the most hazardous to humans. Exposure to spores from Aspergillus fumigatus result in severe allergic reactions in humans. Moreover, Aspergillus flavus produces the mycotoxin aflatoxin which is one of the most potent carcinogens known to man. Also, Aspergillus niger often actively proliferates in the human lung, forming a ball. In certain implementation, the antimold properties of the composition may be against any one or more of Aspergillus fumigatus, Aspergillus flavus, Aspergillus niger or any other species of the Aspergillus genus.

In yet other embodiments, the antimold properties of the composition of the present technology is against the Stachybotrys chartarum, which produces extremely toxic, carcinogenic and immunosuppressive mycotoxins. In further embodiments, the antimold properties of the composition of the present technology may also be against any one or more of black mold, Sladosporium, Cladosporium, Fusarium or Penicillium species.

From another aspect, the present technology relates to a method for the treatment of a surface to prevent fungus growth. The treatment method extends to the removal, prevention and/or prophylactic treatment, reduction, and/or resistance to recurrence of fungus growth on a surface. Generally, the method comprises contacting a surface with an effective amount of any one or more of the antifungal compositions described herein.

In certain embodiments, the surface is any surface of a building, Examples of suitable surfaces include but are not limited to surfaces in schools, hospitals, hotels, residential dwellings, apartments, condominiums, commercial properties, loading docks, offices, modular buildings, transportation systems (e.g., metros, airports, bus stations), and industrial workplaces. The treatment method also may be applied to maritime and aviation vessels containing living quarters, such as in the case of a cruise ship. Various surfaces can be treated using the method and composition of the present technology, including by way of example, the following: metal surfaces, such as steel, aluminum and aluminum alloys, copper and copper alloys, zinc and zinc alloys; plastics, such as polycarbonates, polyvinyl chlorides, polyurethanes, polyolefins, epoxides, nylons; and other non-metal surfaces, such as wood, ceramics, glass, concrete, and the like. Other examples of building materials that may be treated in accordance any one of more of the embodiments described herein include wall board, ventilation and air handling surface areas, AC coils, sub flooring, wood paneling, brick, concrete, OSB board, carpeting, sheetrock, and the like.

The composition of the present technology may be applied to a finished structure or a structure under construction. The composition is useful in the treatment of infected surfaces and the preventative treatment of non-infected surfaces to avoid or suppress future infection. Without wishing to be bound by theory, in the preventative treatment of non-infected surfaces the composition is absorbed into the surface and inhibit growth of fungal matter such as live cells, spores, and mycotoxins associated with molds. For example, when a surface that has no fungal matter is treated with the composition, the surface becomes resistant to mold even if millions of spores and/or live cells are inoculated on said surface. The composition of the present technology is especially useful for preventative treatment of a building under construction, when it is easy to access and pre-treat surface areas to be hidden in the finished building, such as surface areas behind wallboards and sheetrock, surface areas within vents, air conditioning duct, and filters.

In other embodiments, the composition is useful in the treatment of surfaces following a flood resulting from a leaky roof, indoor plumbing, or general building maintenance problems. After a single incident of water damage occurs in a building, molds grow inside walls and then becomes dormant until a subsequent incident of high humidity. In such circumstances, the treatment of contaminated surfaces with the composition of the present technology may be combined with other common remedies such as blockage of the source of moisture, exposure to sunlight, ventilation, installation of non-porous building materials and the like.

In other embodiments, the surface may be any surface in a hospital, such as operating rooms or patient care rooms, as well as other surfaces in research facilities, or facilities in the pharmaceutical industry, such as production areas, where there is zero tolerance for mold. In other embodiments, the surface may be a surface of a medical devices or implanted medical device such as catheters, breathing tubes and prosthetics. In such embodiments, the compositions of the present technology may be applied on the medical device prior to implantation. Alternatively, since compositions of the present technology are natural and nontoxic to humans and animals, the medical device can comprise a reservoir containing the composition, such that the composition can be released in a controlled manner after implantation.

In further embodiments, the surface may be the surface of a plant (e.g., roots, stems, leaves or foliage, originating shoots and the like) and/or seed, and/or soil, an area and an environment in which plants grow or could grow, and/or materials, plants, seeds, soil, surfaces, and/or spaces which are to be protected from attack or infestation by one or more fungus that are harmful to plants. In such embodiments, the method comprises contacting the harmful fungus present on such surfaces, or the surfaces to protected from fungus growth, with an effective amount of any one or more of the compositions described herein. In certain embodiments, the surfaces to be treated or protected from fungus growth include, but are not limited to, those related to agricultural plants. By agricultural plants it is meant plants of which a part (e.g. seeds) or all is harvested or cultivated on a commercial scale or which serve as an important source of feed, food, fibres (e.g. cotton, linen), combustibles (e.g. wood, bioethanol, biodiesel, biomass) or other chemical compounds. Examples of agricultural plants include cereals, e.g. wheat, rye, barley, triticale, oats, sorghum or rice, beet, e.g. sugar beet or fodder beet; fruits, such as pomes, stone fruits or soft fruits, e.g. apples, pears, plums, peaches, almonds, cherries, strawberries, raspberries, blackberries or gooseberries; leguminous plants, such as lentils, peas, alfalfa or soybeans; oil plants, such as rape, oil-seed rape, canola, linseed, mustard, olives, sunflowers, coconut, cocoa beans, castor oil plants, oil palms, ground nuts or soybeans; cucurbits, such as squashes, cucumber or melons; fiber plants, such as cotton, flax, hemp or jute; citrus fruit, such as oranges, lemons, grapefruits or mandarins; vegetables, such as spinach, lettuce, asparagus, cabbages, carrots, onions, tomatoes, potatoes, cucurbits or paprika; lauraceous plants, such as avocados, cinnamon or camphor; energy and raw material plants, such as corn, soybean, rape, canola, sugar cane or oil palm; tobacco; nuts; coffee; tea; bananas; vines (table grapes and grape juice grape vines); hop; turf; natural rubber plants. In other embodiments, the agricultural plants may be field crops such as potatoes, sugar beets, cereals such as wheat, rye, barley, oats, sorghum, rice, corn, cotton, rape, oilseed rape and canola, legumes such as soybeans, peas and field beans, sunflowers, sugar cane, vegetables such as cucumbers, tomatoes, onions, leeks, lettuce and squashes.

In yet further embodiments, the surface to be treated or protected from fungus growth include those related to horticultural plants. By “horticultural plants” it is meant those plants which are commonly used in horticulture—e.g. the cultivation of ornamentals, and are commonly used in gardening, e.g. in parks, gardens and on balconies. Examples for ornamentals are turf, geranium, pelargonia, petunia, begonia and fuchsia.

In other embodiments, the surface to be treated or protected from fungus growth include those related to silvicultural plants. By “silvicultural plant” it is meant trees, more specifically trees used in reforestation or industrial plantations. Industrial plantations generally serve for the commercial production of forest products, such as wood, pulp, paper, rubber tree, Christmas trees, or young trees for gardening purposes. Examples for silvicultural plants are conifers, like pines, in particular Pinus spec, fir and spruce, eucalyptus, tropical trees like teak, rubber tree, oil palm, willow (Salix), in particular Salix spec, poplar (cottonwood), in particular Populus spec, beech, in particular Fagus spec, birch, oil palm and oak.

In yet other embodiments, the surface may be the surface of any article of manufacture which can support fungal growth. Advantageously, the compositions of the present technology are nontoxic and thus safe for humans (including children) and are thus suitable for use on articles of manufacture commonly present in household or other environments occupied by humans and/or animals. Examples of articles of manufacture include for example clothes, toys, furniture and pet articles such as brushes, sponges, combs and the like.

The composition of the present technology may be applied to a surface in any known or suitable manner, including using application techniques such as spraying, atomizing, coating, immersion, immersion ultrasonic, or dipping. According to an embodiment, a standard spray application technique is employed to apply any one or more of the embodiments described herein to the building surface. Standard spray equipment may be used. The composition, in these embodiments, may include particles greater than fifteen (15) microns in size to prevent the occurrence of bacteria and fungi growth and spore germination. According to another embodiment, an atomization fumigation technique is employed for application of the composition. The atomization fumigation application involves misting the composition into particles of about 7 microns to about 15 microns in size and contacting the particles against a building surface. Commercial equipment may be used for spraying or atomizing the composition on a building surface. Especially useful examples of commercial equipment include the Model 7808 NOZ-L-JET atomizer of Fogmasters, Inc. of Deerfield Beach, FIa. and the 534 Specialty Fogger/atomizer of Lafferty Equipment Manufacturing, Inc. of North Little Rock, AR.

In certain embodiments, the composition of the present technology may be left to dry on the surface. In other embodiments, the composition may be left to contact the surface for a given period of time. The contact time between the composition and the surface to be cleaned may be at least 24 hours, more preferably about 24 hours to about 48 hours, although shorter or longer contact times may be selected. The contact time will depend upon several interdependent variables, including the amount and type of fungus and other contamination on the surface to be cleaned, the material composing and porosity of the surface, and the effectiveness of the particular application technique and equipment employed. In these embodiments, removal of the composition following the contact period can be accomplished using known techniques, such as rinsing with water. Treatment may be repeated if desired or necessary. Further, the embodied treatment may be combined with other products and agents. For example, the surface may be pretreated, if desired, to remove excess grime and soil prior to application of the inventive composition. Optionally, the surface may be subjected to pre-treatment or post-treatment procedures, or further treated with other antimicrobial or cleaning products prior or subsequent to removal of the composition. Multiple different antimicrobial compositions may be applied simultaneously or consecutively.

In other embodiments, the present technology relates to a method for preventing and/or inhibiting the growth fungi in a food product. Generally, the method comprises contacting the surface of a food product with an effective amount of any one or more of the antifungal compositions described herein. Examples of surfaces of food products includes surfaces of cans, bottles, skins of dried sausages and the like. In these embodiments, the antimold effect is in respect of a fungus or mold associated with food spoilage or food born disease such as Candida (e.g. C. krusei, C. parapsilosis, C. utilis, C. valida), Dekkera (e.g. D. bruxellensis), Debaryomyces (e.g. D. hansenii), Hanseniaspora (e.g. H. uvarum) Kluyveromyces (e.g. K. loctis), Pichia (P. membranaefaciens), Rhodosporidium, Rhodotorula (Rh mucilaginosa), Saccharomyces (e.g. S. bayanus, S. boulardi, S. carlsbergensis, S. cerevisiae, S. exiguus, S. florentinus, S. unisporus), Zygosaccharonmyces (e.g. Z. rouxii, Z baili) and moulds, especially from the species of Aspergillus (e.g. A. niger, A. restrictus, A. versicolor, A. flavus), Byssochlamys (e.g. B. fulva, B. nivea), Eupenicillium, Eurotium, Fusarium (F. oxysporum, F. graminearum, F. solani), Geotrichum, Mucor, Neosaftorya (e.g. N. fischeri var. fischerl), Penicillium (e.g. P. islandicum, P. citrinum, P. chrysogenum, P. aurantiogriseum, P. brevicompactum, P. camembertii, P. candidum, P. chrysogenum, P. commune, P. corylophilum, P. cyclopium, P. discolor, P. nalgiovense, P. rogueforti), Talaryomyces (e.g. T. macrosporus). In preferred embodiments, the antimold properties of the composition of the present technology may also be against any one or more of black mold, Sladosporium, Cladosporium, Fusarium or Penicillium species. Alternatively, the composition of the present technology may also be incorporated into food products (human or pet and/or animal), such as in beverages and the like.

In other embodiments, the composition of the present technology comprises a mixture of essential oils having a single or a combination of natural monoterpenoid phenols. The composition may be used in pharma industry, construction industry, healthcare industry, manufacturing industries and other industries. In such embodiments, the composition comprises two essential oils; a first essential oil is a tea tree oil, and a second essential oil is a eucalyptus oil. The tea tree oil is present in the composition in an amount of 0.5% by weight plus/minus 0.1% by total weight of the composition. The eucalyptus oil is present in the composition in an amount of 0.5% by weight plus/minus 0.1% by total weight of the composition. In some embodiments, the composition further comprises at least one additive selected from minerals, emulsifiers, defoamers, thickeners, dispersants, stabilizers, suspending agents, adjuvants, preservatives, polymers, acids, bases, dyes, antifreezes, biocides, fillers, wetting agents, and solvents/dilutants. Suitable substances are those which can typically be used for the treatment of surfaces and are compatible with the composition.

In some embodiments, the composition of the present technology further comprises sodium borate (Na₂B₄O₇), wherein sodium borate is present in the composition in an amount of 0.3% by total weight of the composition plus/minus 0.2% by total weight of the composition. In some implementations, the composition of the present technology further comprises an emulsifier such as isodecyl alcohol ethoxylated with 6 to 9 moles of ethylene glycol having a concentration of 2% by weight plus/minus 0.5% by total weight of the composition. The composition of the present technology may further comprise natural polyene biocide nystatin, wherein the concentration of nystatin is 0.03% plus/minus 0.02% by total weight of the composition. The composition may further comprise an organo sulfur acid derived from caprylic acid (octanoic acid) called Alpha Lipoic Acid (ALA). The ALA concentration in the composition is 0.06% plus/minus 0.01% by total weight of the composition. The composition may further comprise an ethyl ester of the lactic acid such as ethyl lactate. The concentration of ethyl lactate is 5.0% by total weight of the composition plus/minus 1.0%. The composition of the present technology may comprise a preservative such as the natural monoterpenoid phenol derivative thymol, wherein thymol is present in the composition in a concentration of 0.5% plus/minus 0.1% by total weight of the composition. Another ingredient that may be present in the composition of the present technology is D isomer of the natural amino acid Phenylalanine (D-Phe). The concentration of D-Phe in the composition is 0.01% plus/minus 0.002%.

### EXAMPLES

### Example Preparation of an Antifungal Composition for Mold Remediation

An antifungal composition for mold remediation was prepared by mixing:
a suitable amount of phenylpropanoid and its synthetic derivative,
a suitable amount of D-isomer of a naturally-occurring aromatic amino acid and its methyl ester derivative,
a suitable amount of a natural monoterpenoid phenol and its iodo-derivative,
a suitable amount of a plant oil extract, and
a suitable amount of a natural polymer with water in an industrial mixer, such that the final proportion of each of the phenylpropanoid and its synthetic derivative, the D-isomer of the naturally-occurring aromatic amino acid and its methyl ester derivative, the natural monoterpenoid phenol and its iodo-derivative, the plant oil extract, and the natural polymer were between about 0.01% and about 5% w/w, between about 0.01% and about 5% w/w, between about 0.1% and about 10% w/w, between about 0.1% and about 10% w/w, and between about 0.1% to about 5% respectively.

Microbiological testing for the antifungal composition for food (Mold Guard® food formula) and for pharmaceutical (Mold Guard® Pharma formula) applications was conducted at Expert Chemical Analysis in San Diego, California. Both compositions were tested against common fungal species and both were proven successful at stopping fungal growth on the treated surface.

The food composition was tested on sterilized sausage casings. The fungal species tested included: Penicillium chrysogenum ATCC 10106; Penicillium funiculosum ATCC 8725; Aspergillus brasiliensis SN26 ATCC 9642; Trichoderma virens; T-1 ATCC 9645; Aureobasidium pullulans; var. Melanigenum; QM 279c ATCC 15233; Telaromyces pinophilus ATCC 9644; and Chaetomium globosum; QM 459 ATCC 6205.

Each sample was tested using Petri dishes (150 mm) that contained sterile nutrient salts agar (pH 6.5) and sterile sausage casings treated with the composition (Mold Guard® food formula). The negative control was two pieces of sterilized sausage casings not treated with Mold Guard® food formula. The sausage casings were embedded in the solidified basal salt agar and inoculated with 100 micro liters of the fungal suspension. Two pieces of sausage casing were used to test the inner and outer surface of the casing. The same treatment was used for the control sample. The seven fungi strains were tested separately. The inoculated test specimens were incubated at 28-30° C. (82-86° F.) with no less than 85% relative humidity. The length of the test was 28 days of incubation as recommended by the ASTM G21 Fungal Defacement Test. The control samples were terminated at 20 days for exhibiting extensive growth. The samples were examined at 4, 12, 20, and 28 days after inoculation for visible effects of fungal growth. The following was observed.
1. The sausage casings treated with Mold Guard® food formula showed no signs of fungal growth 28 days after inoculation.
2. The sausage casing control samples not treated with Mold Guard food formula and inoculated with Penicillium chrysogenum and Penicillium funiculosum were under 60% covered in fungi by day 4 and 100% covered by day 12.
3. The sausage casing control samples not treated with Mold Guard® food formula and inoculated with Aspergillus brasiliensis and Trichoderma virens was 100% covered in fungi by day 4.
4. The sausage casing control samples not treated with Mold Guard® food formula and inoculated with Aureobasidium pullulans; var. melanigenum, Talaromyces pianophiles, and Chaetomium globosum was 30% covered by day 4 and 100% covered by day 12.

The sausage casings samples treated with Mold Guard® food formula prevented fungi growth for 28 days (required by the ASTM G21 Fungal Defacement Test) at 28-30° C. (82-86° F.) with no less than 85% relative humidity. Our data showed that Mold Guard® food formula can prevent fungi growth on a treated surface.

The pharmaceutical formula (Mold Guard® Pharma formula) was tested on pieces of dry wall. The fungal species tested included: Penicillium chrysogenum ATCC 10106; Trichoderma virens; T-1 ATCC 9645; Aureobasidium pullulans; var. Melanigenum; QM 279c ATCC 15233; Telaromyces pinophilus ATCC 9644; Chaetomium globosum; QM 459 ATCC 6205.

Each sample was tested using Petri dishes (150 mm) that contained sterile nutrient salts agar (pH 6.5) and sterile drywall (2' diameter) treated with the composition (Mold Guard® Pharma formula). The negative control was two pieces of sterilized dry wall not treated with Mold Guard® Pharma formula. The 2-inch drywall pieces were embedded in the solidified basal salt agar and inoculated with 100 micro liters of the fungal suspension. The same treatment was used for the control sample. The five fungi strains were tested separately. The inoculated test specimens were incubated at 28-30° C. (82-86° F.) with no less than 85% relative humidity. The length of the test was 28 days of incubation as recommended by the ASTM G21 Fungal Defacement Test. The control samples were terminated at 20 days for exhibiting extensive growth. The samples were examined at 4, 12, 20, and 28 days after inoculation for visible effects of fungal growth. The following was observed.
1. The dry wall treated with Mold Guard® Pharma formula shows no signs of fungal growth 28 days after inoculation.
2. The dry wall control samples not treated with Mold Guard® Pharma formula and inoculated with Penicillium chrysogenum, Telaromyces pinophilus, and Chaetomium globosum were under 60% covered in fungi by day 4 and 100% covered by day 12.
3. The dry wall control samples not treated with Mold Guard® Pharma formula and inoculated with Trichoderma virens was 100% covered in fungi by day 4.
4. The dry wall control sample not treated with Mold Guard® Pharma formula and inoculated with Aureobasidium pullulans; var. melanigenum was under 60% covered by day 4 and more than 90% covered by day 12.

The drywall samples treated with Mold Guard® prevented fungal growth for 28 days (required by the ASTM G21 Fungal Defacement Test) at 28-30° C. (82-86° F.) with no less than 85% relative humidity. This data shows that the compositions of the present technology can prevent fungi growth on a treated surface.

Variations and modifications will occur to those of skill in the art after reviewing this disclosure. The disclosed features may be implemented, in any combination and subcombinations (including multiple dependent combinations and subcombinations), with one or more other features described herein. The various features described or illustrated above, including any components thereof, may be combined or integrated in other systems. Moreover, certain features may be omitted or not implemented. Examples of changes, substitutions, and alterations are ascertainable by one skilled in the art and could be made without departing from the scope of the information disclosed herein.

It should be appreciated that the present technology is not limited to the particular embodiments described and illustrated herein but includes all modifications and variations falling within the scope of the present technology as defined in the appended claims.

1. An antifungal composition comprising:
i) at least one naturally-occurring compound of the phenylpropanoid family;
ii) at least one synthetic derivative of a compound of the phenylpropanoid family;
iii) at least one D-isomer of a naturally-occurring aromatic amino acid;
iv) at least one synthetic derivative of a D-isomer of a naturally-occurring aromatic amino acid;
v) at least one natural monoterpenoid phenol; and
vi) at least one halogenated derivative of a natural monoterpenoid phenol.

2. The antifungal composition of claim 1, wherein the proportion of the at least one naturally-occurring compound of the phenylpropanoid family, and the at least one synthetic derivative of a compound of the phenylpropanoid family in the composition, is between about 0.01% and about 5% w/w.

3. The antifungal composition of claim 1, wherein the proportion of the at least one D-isomer of a naturally-occurring aromatic amino acid and the at least one synthetic derivative of a D-isomer of a naturally-occurring aromatic amino acid in the composition, is between about 0.01% and about 5% w/w.

4. The antifungal composition of claim 1, wherein the proportion of the at least one natural monoterpenoid phenol and the at least one halogenated derivative of a natural monoterpenoid phenol in the composition is between about 0.1% and about 10% w/w.

5. The antifungal composition of claim 1, wherein the at least one naturally-occurring compound of the phenylpropanoid family is selected from D-Phenylalanine, D-Tyrosine, cinnamic acid, coumaric acid, caffeic acid, ferulic acid, 5-hydroxyferulic acid, sinapic acid, cinnamaldehyde, umbelliferone chalcone, eugenol, chavicol, safrole, estragole, resveratrol, apiole, asarone, dillapiole, elemicin, estragole, methyl eugenol, myristicin, monolignols, lignins, suberins and stilbenoids, and combinations thereof.

6. The antifungal composition of claim 1, wherein the at least one naturally-occurring compound of the phenylpropanoid family is selected from D-Phenylalanine, D-Tyrosine, Cinnamic acid and Eugenol, and combinations thereof.

7. The antifungal composition of claim 1, wherein the at least one synthetic derivative of a compound within the phenylpropanoid family is selected from at least one synthetic phenylalanine derivative, and at least one synthetic tyrosine derivative.

8. The antifungal composition of claim 7, wherein the at least one synthetic phenylalanine derivative is selected from D-phenylalanine methyl ester, 2-nitro-D-phenylalanine, pentafluoro-D-phenylalanine, and 2-chloro-D-phenylalanine, and combinations thereof.

9. The antifungal composition of claim 7, wherein the at least one synthetic tyrosine derivative is selected from N-acetyl-D-tyrosine, D-tyrosine methyl ester, and D-tyrosine ethyl ester and combinations thereof.

10. The antifungal composition of claim 1, wherein the at least one D-isomer of a naturally-occurring aromatic amino acid is selected from D-phenylalanine (DPA), D-tryptophan, D-tyrosine and D-histidine.

11. The antifungal composition of claim 1, wherein the at least one synthetic derivative of a D-isomer of a naturally-occurring aromatic amino acid is a methyl ester of the at least one D-isomer of the naturally-occurring aromatic amino acid.

12. The antifungal composition of claim 1, wherein the at least one D-isomer of a naturally-occurring aromatic amino acid is D-tyrosine.

13. The antifungal composition of claim 12, wherein the at least one synthetic derivative of a D-isomer of a naturally-occurring aromatic amino acid is D-Tyrosine-methyl-ester.

14. The antifungal composition of claim 1, wherein the at least one D-isomer of a naturally-occurring aromatic amino acid is DPA.

15. The antifungal composition of claim 14, wherein the at least one synthetic derivative of a D-isomer of a naturally-occurring aromatic amino acid is D-Phenylalanine-methyl-ester.

16. The antifungal composition of claim 1, wherein the at least one natural monoterpenoid phenol is selected from carvacrol, thymol, eucalyptol and eugenol.

17. The antifungal composition of claim 1, wherein the at least one natural monoterpenoid phenol is thymol.

18. The antifungal composition of claim 1, wherein the at least one halogenated derivative of a natural monoterpenoid phenol is selected from 4-chlorothymol, 4-iodothymol, bromothymol, chlorocarvacrol, iodocarvacrol, bromocarvacrol, chloroeugenol, iodoeugenol and bromoeugenol.

19. The antifungal composition of claim 1, wherein the at least one halogenated derivative of a natural monoterpenoid phenol is 4-chlorothymol.

20. The antifungal composition of claim 1, wherein the at least one halogenated derivative of a natural monoterpenoid phenol is 4-iodothymol.

21.-60. (canceled)
